(19) United States
(12) Patent Application Publication
(10) Pub. No.: US 2025/0281161 A1
(43) Pub. Date: Sep. 11, 2025

(54) ULTRASONIC SYSTEM AND METHOD FOR MEDICAL INSTRUMENT LOCALIZATION AND POSITIONING GUIDANCE

(71) Applicant: Rivanna Medical, Inc., Charlottesville, VA (US)

(72) Inventors: Kathryn Ozgun, Charlottesville, VA (US); Paul Sheeran, Charlottesville, VA (US); Adam Dixon, Charlottesville, VA (US); Frank William Mauldin, Jr., Troy, VA (US)

(21) Appl. No.: 18/913,791

(22) Filed: Oct. 11, 2024

Related U.S. Application Data
(63) Continuation of application No. 17/950,399, filed on Sep. 22, 2022.
(60) Provisional application No. 63/246,859, filed on Sep. 22, 2021.
(60) Provisional application No. 63/543,638, filed on Oct. 11, 2023.

(51) Int. Cl. A61B 8/00; A61B 8/08; A61B 90/00

(52) U.S. Cl. CPC A61B 8/5253; A61B 8/0841; A61B 8/4461; A61B 8/4477; A61B 8/483; A61B 90/37

(57) ABSTRACT

Systems and methods for ultrasound-guided placement of a medical instrument, such as a needle, that enhance the medical instrument's visibility and provide real-time display feedback on the instrument's location, trajectory, or combination of location and trajectory.

### CROSS REFERENCE TO RELATED APPLICATIONS

The present application relies on the disclosures of and claims priority to and the benefit of the filing date of U.S. application Ser. No. 17/950,399, filed Sep. 22, 2022, which relies on the disclosures of and claims priority to and the benefit of the filing date of U.S. application Ser. No. 63/246,859, filed Sep. 22, 2021. The present application also relies on the disclosures of and claims priority to and the benefit of the filing date of U.S. application Ser. No. 63/543,638, filed Oct. 11, 2023. The disclosures of the above applications are hereby incorporated by reference herein in their entireties.

### STATEMENT REGARDING FEDERALLY SPONSORED RESEARCH

This invention was made with government support under Grant No. R44NS120798 awarded by the National Institutes of Health (NIH) National Institute of Neurological Disorders and Stroke (NINDS). The government has certain rights in the invention.

### TECHNICAL FIELD

This invention is related to medical instrument guidance systems and methods, and more particularly to methods of ultrasonic guidance to assist insertion of medical instruments, such as needles, toward a desired anatomical target.

### BACKGROUND OF THE INVENTION

The present invention relates to ultrasound-guided medical needle placement. Many medical procedures involve the insertion of a medical instrument, such as a medical needle, toward a target anatomical region, encompassing activities such as biopsies, drug administration, and vascular access. Ultrasound-based needle guidance requires expertise in ultrasound image interpretation and in managing the needle's orientation relative to the ultrasound imaging plane.

Medical instrument placement procedures to place medical needles under ultrasonic guidance are often conducted using a longitudinal, or in-plane, trajectory to permit visualization of the full needle shaft as it advances toward the target site. Longitudinal needle insertions involve positioning the needle entry point adjacent to the probe, using a shallow angle of approach to reach the target anatomy. However, in certain types of procedures, a steeper angle of approach is preferred due to anatomical constraints. One method of placing needles using a steep angle of approach is to use a transverse, or out-of-plane, trajectory relative to the imaging plane; the primary disadvantage of this technique is that only a short segment of the needle is visualized. Alternatively, needles may be inserted in-plane at a steep angle, but this technique is particularly challenging. When the needle is aligned parallel to the ultrasound beam, the reflectivity is low, which results in diminished needle visibility. The lack of visibility reduces the operator's ability to ascertain the needle's trajectory and may result in inaccurate needle placement. Errant needle placement prolongs procedure time, increases needle redirections, and decreases patient satisfaction.

Techniques to detect needles within ultrasound images consider characteristics such as the needle's shape, motion, intensity, or location. Most methods for needle guidance on commercial ultrasound systems enhance the appearance of a detected needle shaft by increasing the brightness of pixels containing the needle segment relative to the surrounding tissue, or by encoding pixels containing the needle segment with a color that differs from the surrounding tissue. While these methods are effective to localize needles in soft tissue anatomies placed at shallow angles, enhancement of the needle shaft is less successful for guiding needle insertions at steep angles. Therefore, there exists a need for a real-time technique that provides feedback about a needle's trajectory relative to the target anatomy, particularly for in-plane needle placements with a steep angle of approach.

To overcome the limitations of current state of the art approaches to medical needle guidance procedures, the present invention describes unique ultrasound systems and methods to acquire ultrasound images on a multi-array ultrasound probe, detect a medical instrument inserted in patient anatomy, enhance visualization of the medical instrument, and provide indicators to improve procedure guidance on a display unit. The ultrasound system and methods can be used image anatomy and provide procedure guidance in either 2-dimensional or 3-dimensional image data. A physical gap between the ultrasound arrays enables in-plane needle insertion during contemporaneous image acquisition. Image and signal processing methods are used to detect motion and morphology-based characteristics of the medical instrument relative to surrounding biological tissue and enhance visibility of the medical instrument in the displayed images. Novel display formats that more intuitively represent the spatial relationship of the ultrasound probe, operator, and patient are described. Various preferred embodiments of the invention are described herein.

Related art describes apparatus to facilitate interventional procedures involving medical instruments.

U.S. Pat. No. 11,529,115B2, hereby incorporated by reference herein, describes a system comprising an ultrasound probe and puncture needle, the ultrasound probe having a wedge-shaped configuration so that a single ultrasound transducer array housed inside the probe is tilted at an angle relative to the body, providing ultrasound probe configured to be angled relative to patient anatomy. As it relates to the current invention described herein, among other distinctions, the system of U.S. Pat. No. 11,529,115B2 does not support two or more arrays and does not permit an in-plane midline needle trajectory.

PCT Application No. PCT/CA2009/001700, hereby incorporated by reference herein, describes an ultrasound imaging and medical instrument guiding apparatus comprising two ultrasound probes configured on a mount to acquire distinct 3-dimensional images of overlapping volumes and a positionable medical instrument guide that allows propagation of the medical instrument into the overlapped region of the imaging volumes from the two ultrasound probes. However, the configuration of PCT/CA2009/001700 differs from the current invention described herein, and in embodiments of the current invention described herein, the apparatus comprises methods to detect and enhance visibility of an inserted medical instrument on the basis of relative motion and properties of morphology compared to surrounding biological tissue, which again, among other reasons, is unlike PCT/CA2009/001700.

U.S. application Ser. No. 06/396,784, hereby incorporated by reference herein, describes an ultrasonic probe for use in needle insertion procedures, the ultrasonic probe including a support having an array of ultrasonic transducer elements lying flatwise on the front end and a groove in the support for guiding the needle. In the ultrasonic probe of U.S. application Ser. No. 06/396,784, the groove forms an opening at the front end of the support, and one or more transducer elements are located adjacent the opening of the groove and between the other transducer elements, thus leaving no blank space on the front end of the support. However, the configuration of U.S. application Ser. No. 06/396,784 differs from the current invention described herein, and in embodiments of the current invention described herein, the apparatus comprises methods to detect and enhance visibility of an inserted medical instrument on the basis of relative motion and properties of morphology compared to surrounding biological tissue, which again, among other reasons, is unlike U.S. application Ser. No. 06/396,784.

U.S. Pat. No. 4,387,721A, hereby incorporated by reference herein, describes an ultrasonic probe comprising two flat ultrasound transducer arrays having a groove between the two flat ultrasound transducer arrays, and further requires a cannula (needle) placed parallel to the primary axis of the groove. In differentiation with the ultrasonic probe of U.S. Pat. No. 4,387,721A, the current invention comprises ultrasound transducer arrays that produce overlapping images and components providing for three-dimensional viewing. Furthermore, in embodiments of the current invention described herein, the apparatus comprises methods to detect and enhance visibility of an inserted medical instrument on the basis of relative motion and properties of morphology compared to surrounding biological tissue, which again, among other reasons, is unlike U.S. Pat. No. 4,387,721A, by way of example, and is overall an improvement over U.S. Pat. No. 4,387,721A, as well as the other related art.

U.S. Pat. No. 4,489,730A hereby incorporated by reference herein, describes an ultrasonic transducer probe comprising a flat ultrasound transducer array with a gap that can receive a removeable wedge-shaped cannula (needle) adapter. In differentiation with the ultrasonic probe of U.S. Pat. No. 4,489,730A, the current invention described herein comprises ultrasound transducer arrays that produce overlapping images and components providing for three-dimensional viewing. Finally, the current invention comprises methods to detect and enhance visibility of an inserted medical instrument on the basis of relative motion and properties of morphology compared to surrounding biological tissue, which again, among other reasons, is unlike U.S. Pat. No. 4,489,730A, by way of example, and is overall an improvement over U.S. Pat. No. 4,489,730A, as well as the other related art.

U.S. Pat. No. 4,289,139A hereby incorporated by reference herein, describes an ultrasonic transducer probe comprising ultrasonic transducer elements arranged proximate to a surface that is positioned on the body surface of a subject, further comprising a shaped cavity that provides a guide block for a cannula (needle) while also allowing for removal of the ultrasonic transducer probe from the inserted canula, the guide block being sterilizable after removable. In differentiation with the ultrasonic probe of U.S. Pat. No. 4,289,139A, the current invention comprises ultrasound transducer arrays that produce overlapping images and components providing for three-dimensional viewing. Finally, the current invention comprises methods to detect and enhance visibility of an inserted medical instrument on the basis of relative motion and properties of morphology compared to surrounding biological tissue, which again, among other reasons, is unlike U.S. Pat. No. 4,289,139A, by way of example, and is overall an improvement over U.S. Pat. No. 4,289,139A, as well as the other related art.

GB0307311A, hereby incorporated by reference herein, describes an ultrasound probe comprising a housing and guide for needle insertion, the guide comprising a channel located between ultrasound transducers in the housing. In differentiation with the ultrasonic probe of GB0307311A, the current invention described herein comprises methods to detect and enhance visibility of an inserted medical instrument on the basis of relative motion and properties of morphology compared to surrounding biological tissue, which again, among other reasons, is unlike GB0307311A, by way of example, and is overall an improvement over GB0307311A, as well as the other related art.

U.S. Pat. No. 11,877,888B2, hereby incorporated by reference herein, describes a system comprised of, among other elements, an ultrasound probe housing comprising multiple arrays and two or more graphical overlays, one overlay being in the place of the needle and at least one over an anatomical target. In differentiation with the ultrasonic system of U.S. Pat. No. 11,877,888B2, the current invention described herein comprises systems and methods to detect and enhance visibility of an inserted medical instrument on the basis of relative motion and properties of morphology of the medical instrument compared to surrounding biological tissue. Instead of replacing the needle and/or target with indicator(s), the current invention avoids obstructing the original image information and, in one embodiment, provides adjacent instructional information. Whereas the ultrasonic system of U.S. Pat. No. 11,877,888B2 processes the images to identify the presence of a needle and an anatomical target in a binary manner, the current invention teaches techniques to enhance native image pixel locations of these objects within the ultrasound image. The ultrasound probe of the current invention is further differentiated from the ultrasonic system of U.S. Pat. No. 11,877,888B2 where the gap between the two or more arrays is at least 1 mm, permits an in-plane insertion of the medical instrument, and permits steering of the ultrasound beam inward toward the needle. Furthermore, the current invention teaches alteration of the image pixels containing a medical instrument to improve native contrast of the medical instrument, which again, among other reasons, is unlike U.S. Pat. No. 11,877,888B2, by way of example, and is overall an improvement over U.S. Pat. No. 11,877,888B2, as well as the other related art.

U.S. application Ser. No. 11,432,801B2, hereby incorporated by reference herein, describes a system comprising an ultrasound probe, the ultrasound probe comprising two ultrasound transducers arranged at an angle that transmit sound waves to create an overlapping imaging region for 2D and 3D imaging, and a detachable needle guide disposed between the two transducers that extends toward a target location in the overlapping imaging region. In differentiation with the ultrasonic probe of U.S. application Ser. No. 11,432,801B2, the current invention described herein comprises methods to detect and enhance visibility of an inserted medical instrument on the basis of relative motion and properties of morphology compared to surrounding biological tissue, which again, among other reasons, is unlike U.S. application Ser. No. 11,432,801B2, by way of example, and is overall an improvement over U.S. application Ser. No. 11,432,801B2, as well as the other related art.

### SUMMARY OF THE INVENTION

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of this disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

The present invention overcomes limitations of existing ultrasound guidance by providing a system that enables a user to observe steep-angle needle trajectories in real-time or substantially real-time. In embodiments, an ultrasound probe may be used to collect a series of ultrasound images of a patient's anatomy. The collected ultrasound images can be processed by a module configured to detect the presence of trajectory of a needle. The trajectory can be conveyed to the user using an indicator overlaid on a real-time ultrasound image.

In embodiments, the system employs an ultrasound probe with a dual-array geometry that supports a steep-angle needle trajectory through the center of the probe silhouette. The system may act as or comprise a needle guide apparatus to constrain the needle insertion to a range of prescribed trajectories. The constrained range of needle trajectories may serve to improve image processing.

In embodiments, the system employs an indicator that conveys the alignment of a needle's trajectory relative to a designated anatomical location, which enables needle guidance for clinical procedures that target specific anatomy.

Another aspect of the invention overcomes limitations of existing ultrasound processing approaches by inferring the position of a needle using spatial and temporal characteristics to provide real-time feedback for the purpose of guiding a needle. The combination of spatial and temporal measurements can be critical for steep-angle needle placements due to compromised reflectivity and the possibility for the needle to be stationary.

In embodiments, the currently described method and system can comprise capturing a plurality of ultrasound image frames in a memory buffer, which enables detection of steep-angled needles using spatial and temporal characteristics. A processor can be configured to measure the detection confidence and trajectory of a candidate needle based on a weighted combination of measurements. The estimate may be further refined using a probabilistic filter configured to predict the future path of the needle.

In embodiments, the motion field within the imaging plane is determined from ultrasound image frames using probabilistic methodologies. Candidate needles may be identified using the detection of motion along a specified trajectory range. In addition, morphological processing may be applied to one or more images to identify clusters of pixels that exhibit the visual appearance of a needle.

This overview is intended to provide an overview of subject matter of the present patent application. It is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings, in which:

FIGS. 1A and 1B are schematic illustrations of exemplary multi-array ultrasound probes designed to accommodate placement of a medical instrument.

FIG. 2 is a schematic illustration of an exemplary ultrasound imaging and medical guidance system comprised of an ultrasound probe, a processor, and a display unit.

FIGS. 3A-3C are schematic illustrations of exemplary multi-array ultrasound probes designed to accommodate placement of a medical instrument.

FIG. 4 is a diagram of an exemplary signal processing methodology to process a sequence of ultrasound images for the purpose of enhancing pixels containing a medical instrument.

FIGS. 5A and 5B are exemplary representations of an ultrasound images from the current invention.

FIG. 6 is an exemplary graphical representation of an exemplary ultrasound imaging and medical guidance system comprised of an ultrasound probe, a processor, and a display unit.

FIG. 7 is an exemplary graphical representation of an exemplary ultrasound imaging and medical guidance system comprised of an ultrasound probe, a processor, and a display unit.

FIG. 8 is a schematic illustration of an exemplary ultrasound image display including graphical indicators and intensity enhancement of pixels containing a medical instrument.

FIG. 9 is a block diagram of an exemplary signal processing methodology to predict the trajectory of a medical instrument.

### DETAILED DESCRIPTION OF VARIOUS EMBODIMENTS OF THE INVENTION

Ultrasound image guidance systems are used to support a variety of medical or clinical applications. The preferred embodiments herein describe an ultrasound image guidance system to facilitate placement of a medical instrument at or within a region of target anatomy. Those skilled in the art will appreciate that the present invention may be used to guide a variety of medical instruments including, but not limited to, needle, a catheter, trocar, ablation instrument, cutting instrument, or therapy applicator. The objective of the ultrasound system is to produce an image that can be used for real-time guidance. The term real-time is used herein to encompass processing that occurs at a sufficiently rapid rate to be perceived by a user as occurring in the moment.

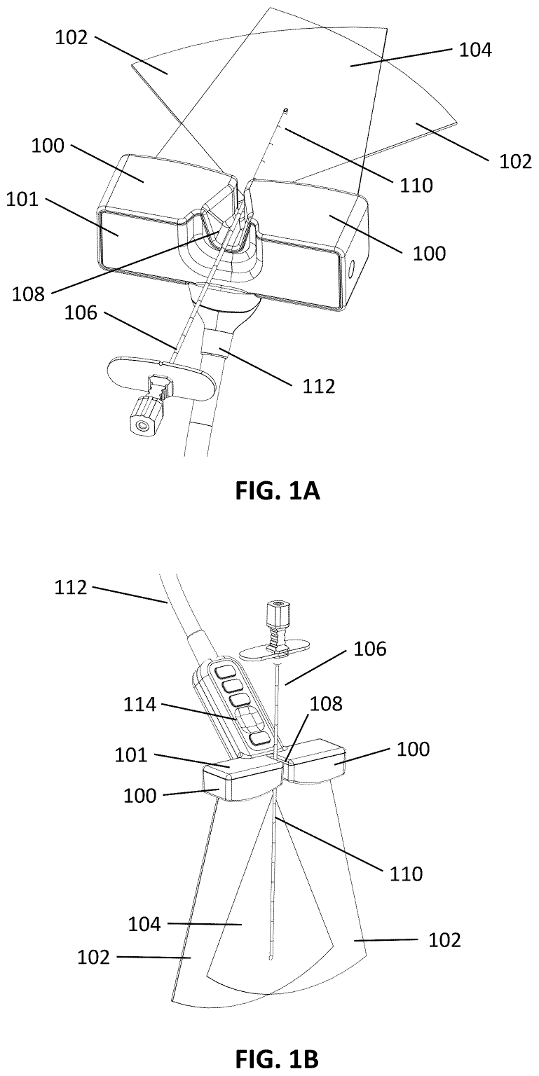

In one embodiment, a multi-array ultrasound imaging probe is depicted in FIG. 1A. The ultrasound probe is comprised of two ultrasound arrays 100 disposed in a probe housing 101. Each of the two arrays transmits and receives ultrasonic pressure waves in an imaging sector 102 that includes a region of acoustic overlap 104. The ultrasonic pressure waves are converted into beamformed ultrasound data by the ultrasound system to provide real-time anatomical visualization and perform image and signal processing to detect the medical instrument, enhance regions of the ultrasound data corresponding to the medical instrument, and provide real-time feedback on the localization of the medical instrument. The configuration of the ultrasound arrays 100 and probe housing 101 allows for a medical instrument 106 to pass through a physical gap 108 in the probe housing 101 such that the medical instrument 106 can be inserted in-plane along the midline of the probe housing 101 through the physical gap 108. The portion of the medical instrument that is inserted into the patient anatomy 110 produces differences in relative motion and properties of morphology compared to the surrounding biological tissue, which can be assessed independently from each of the two ultrasound arrays 100 in the region of acoustic overlap 104, or from one of the two ultrasound arrays 100 in the extended field of acoustic sensitivity. In one embodiment, the ultrasound probe transfers ultrasound data to the ultrasound system by an ultrasound cable 112. In other embodiments, the probe may transfer ultrasound data to the ultrasound system through a Bluetooth or other wireless connection format. FIG. 1B depicts an embodiment of the ultrasound imaging probe wherein the probe housing 101 is extended to incorporate an on-probe electronics unit 114. In embodiments, the on-probe electronics unit 114 comprises electronic components to register changes in spatial position and/or angulation of the ultrasound probe for the purposes of reconstructing ultrasound data into 3-dimensional images, or for altering the orientation of the graphics on a display unit, or for combinations thereof. In embodiments, the electronic components contained in the on-probe electronics unit 114 comprises electronic elements that provide haptic feedback to the operator, such vibration or force, to indicate aspects of procedure guidance. In embodiments, the electronic components contained in the on-probe electronics unit 114 comprises electronic elements such as buttons, microphones, and pressure sensors to receive input from an operator that changes the operational state of the ultrasound system. In a preferred embodiment, the physical gap 108 constrains the range of acceptable medical instrument trajectories within the ultrasound imaging plane. Thus, a set of possible medical instrument trajectories can be determined a priori and used as input to a processor. As an example, in embodiments, the needle may be placed through the needle guide fitted to the ultrasound probe previously disclosed in the co-pending U.S. patent application U.S. 20230090966A1 entitled “Ultrasound-based imaging dual-array probe apparatus and system”, which is incorporated by reference herein, to require that a needle follows a steep-angle trajectory through the center of the probe silhouette.

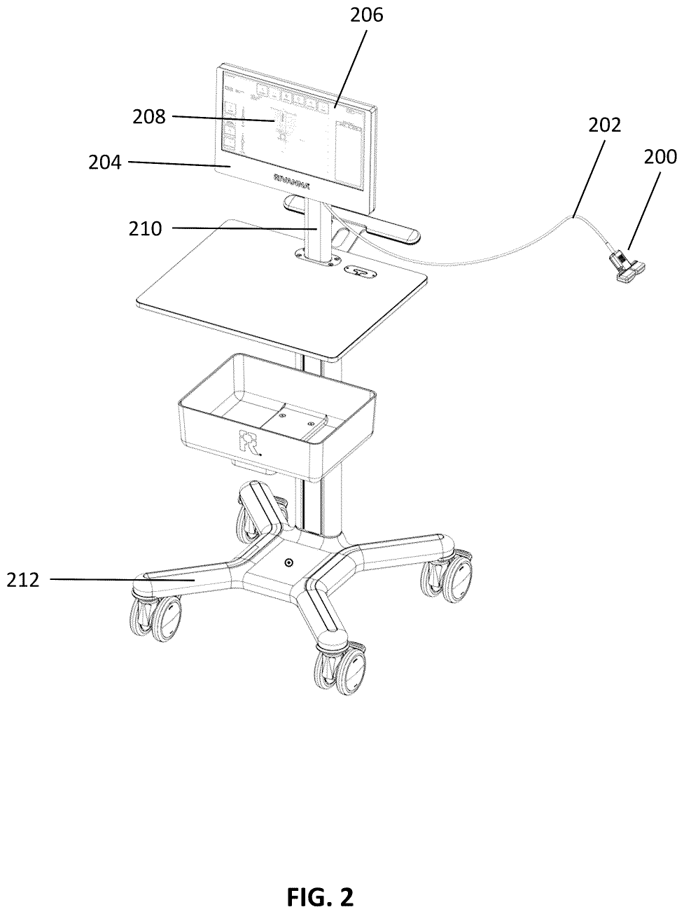

In one embodiment depicted in FIG. 2, the ultrasound system is comprised of an ultrasound probe 200 connected via an ultrasound cable 202 to a computing unit 204 containing an embedded processor and a display unit 206. The embedded processor is used to perform the ultrasound signal and image processing steps required to form ultrasound images as well as to detect the medical instrument and convey aspects of the medical instrument to the user during procedure guidance. Details regarding ultrasound acquisition and image reconstruction, which may be adapted to the present embodiment, are described by co-pending U.S. patent application Ser. No. 63/452,874 entitled “Medical Ultrasound Image Processing Apparatus”, the disclosure of which is hereby incorporated by reference. In embodiments, the display unit 206 displays a graphical representation 208 of ultrasound images in real-time or substantially real-time for the purposes of anatomical imaging and procedure guidance. In embodiments, the display unit 206 displays a graphical representation of a 3-dimensional image acquired by the ultrasound probe 200. In embodiments, the graphical representation 208 contains pixels modified to enhance the visibility of the needle contained within the ultrasound images, such as by modifying one or more of the following relative to surrounding biological tissue: (a) the intensity of pixels containing the medical instrument, (b) the hue of pixels containing the medical instrument, (c) the saturation of pixels containing the medical instrument, (d) the luminance of pixels containing the medical instrument, or (e) combinations thereof. In embodiments, the graphical representation 208 contains one or more of: (a) indicators to convey spatial relationships between the probe geometry and the expected needle path, (b) indicators to convey the measured trajectory of the medical instrument, (c) indicators to represent a comparison of the measured trajectory of the medical instrument and an expected trajectory, or (d) combinations thereof. In embodiments, the computing unit 204 is attached to a mounting pole 210 connected to a medical cart 212 with an independent wheelbase to operate as a standalone unit. In embodiments, the computing unit 204 is connected to a mounting pole 210 that attaches to a multi-purpose medical cart, such as those used for epidural anesthesia administration.

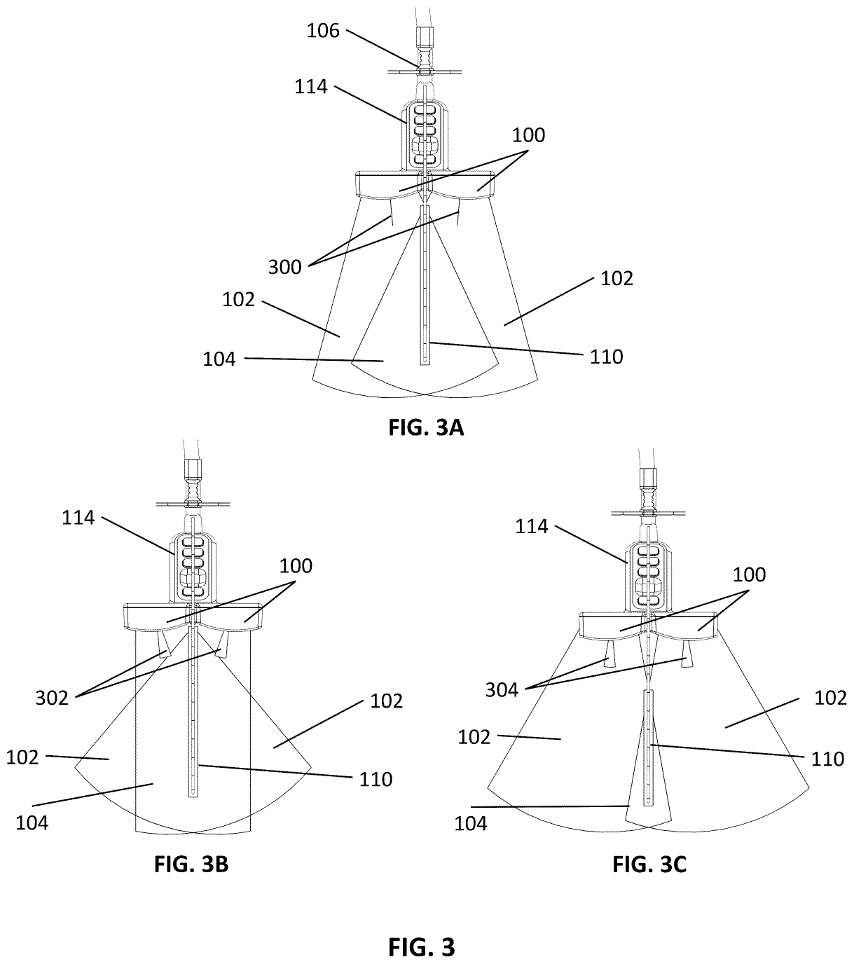

In an embodiment depicted in FIGS. 3A-3C, the ultrasound probe electronics are configured to transmit and receive ultrasound energy at selectable insonation angles relative to each ultrasound array's 100 central axis to generate ultrasound data containing the patient anatomy and the medical instrument 110 within the patient anatomy. FIG. 3A depicts ultrasound imaging sectors 102 that are obtained by transmitting and receiving ultrasound energy along each ultrasound array's 100 central axis. FIG. 3B depicts ultrasound imaging sectors 102 that are obtained by transmitting and receiving ultrasound energy at an inward-steered insonation angle relative to each ultrasound array's 100 central axis, with an acoustic overlap 104 differing from FIG. 3A. FIG. 3C depicts ultrasound imaging sectors 102 that are obtained by transmitting and receiving ultrasound energy at an outward-steer insonation angle relative to each ultrasound array's 100 central axis, with an acoustic overlap 104 differing from FIGS. 3A and 3B. In embodiments, ultrasound data are collected at two or more unique angles for one or more of the ultrasound arrays to quantify motion of the medical instrument 110 relative to surrounding biological tissue, properties of morphology of the medical instrument 110 relative to surrounding biological tissue, or combinations thereof as a function of insonation angle. In embodiments, the ultrasound array can be configured to generate high intensity acoustic radiation force to effectuate motion of the surrounding tissue to further differentiate tissue from a medical instrument and/or rigid biological tissues (e.g. bone or ligament). In embodiments, the ultrasound data collected from each ultrasound array 100 and insonation angle are processed independently to quantify relative motion and/or properties of morphology of the medical instrument. In embodiments, the ultrasound data collected from each ultrasound array 100 and insonation angle are combined, such as through averaging, spatial compounding, or other mathematical operations that would be understood to one practiced in the art, to quantify relative motion and/or properties of morphology of the medical instrument. In embodiments, a subset of the ultrasound data from each ultrasound array and insonation angle are used for anatomical imaging and a subset of the ultrasound data from each ultrasound array and insonation angle are used for quantification of motion and/or properties of morphology of the medical instrument.

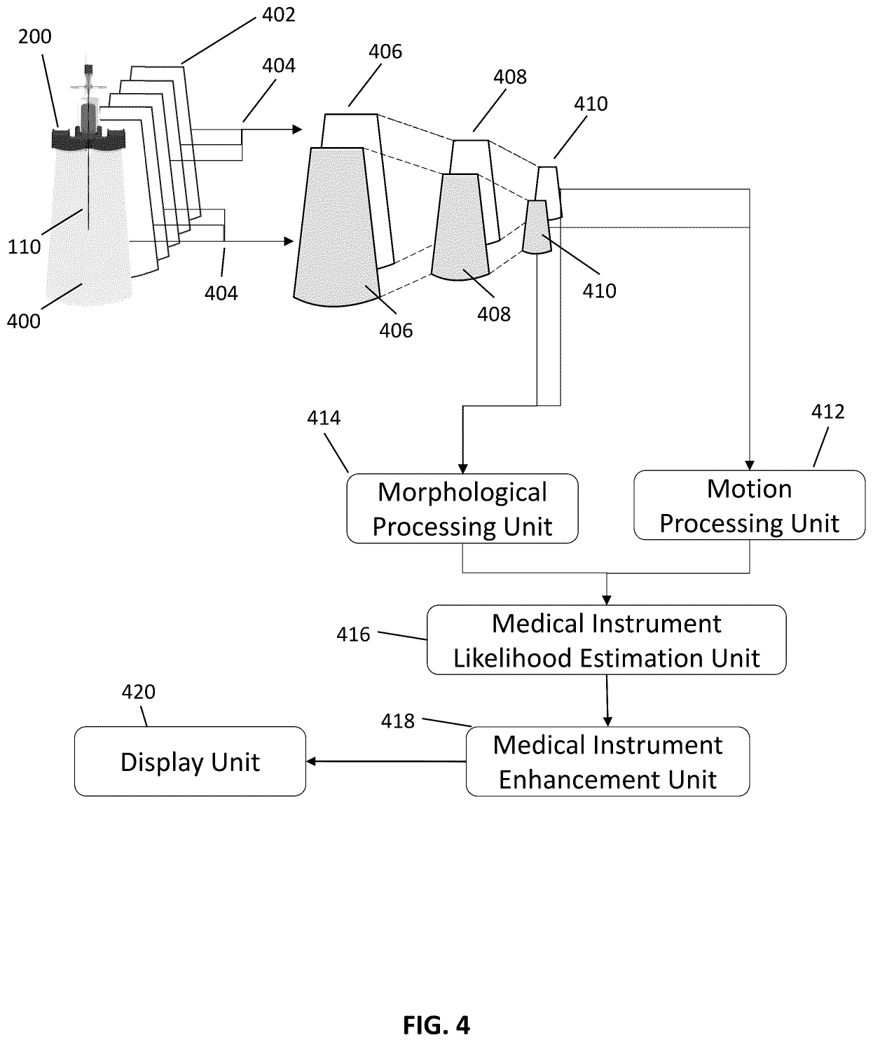

As depicted in FIG. 4 and in operation according to the embodiments, the ultrasound probe 200 transmits and receives ultrasound energy from two or more arrays to image a region 400 containing patient anatomy for the purpose of guiding a medical instrument 110. The ultrasound system processes the ultrasound data from the ultrasound probe 200 to form data frames. One or more data frames are assembled to form an image dataset 402 that is analyzed to determine a likelihood that pixels within the image dataset 402 contain a medical instrument 110, such as through analysis of relative motion of the medical instrument 110 relative to surrounding biological tissue and/or properties of morphology of the medical instrument 110 relative to surrounding tissue. In embodiments, two or more images undergo consolidation 404, such as through averaging, spatial compounding, or other mathematical operations that would be understood to one practiced in the art to produce consolidated data frames 406 prior to quantifying motion and/or properties of morphology. In embodiments, motion and/or properties of morphology are quantified from data frames at two or more resolution scales 408, 410 by modifying the spatial sampling of the data frames 402 or consolidated data frames 406 through multi-scale signal processing, such as Gaussian pyramid processing, Laplacian pyramid processing, or other mathematical operations that would be understood to one practiced in the art.

In embodiments, motion processing unit 412 processes ultrasound datasets, which may comprise the source image dataset 402, consolidated data frames 406, or multi-scale datasets 408, 410. The motion processing unit 412 estimates the motion between two or more data frames in the input dataset and differentiates motion likely to originate from a medical instrument relative to surrounding biological tissue, such as by differentiating highly directional and highly localized motion relative to global motion properties in the dataset. The considered images may be any representative 2D images or 3D volumes obtained by the ultrasound system. In embodiments the motion processing comprises one or more of: (a) ultrasound color Doppler, (b) ultrasound power Doppler, (c) optical flow processing, (d) singular value decomposition processing, or (e) combinations thereof. The motion processing unit 412 may be analytical in nature or may be a machine learning network that is trained to estimate motion. The trained machine learning model may be comprised of one or more generative adversarial networks (GANs), or any other machine learning model capable of generating 2D or 3D motion estimates. The machine learning model architecture may comprise a U-Net, spatially aware autoencoders, context aware autoencoders, long-term short memory (LTSM), and/or recurrent neural networks (RNNs), as well as any other model architecture element advantageous for the purpose of generating motion estimates from input images. In an exemplary embodiment, the machine learning model is trained through a generative adversarial network (GAN) architecture that generates a motion estimate from an input sequence of ultrasound images by iteratively refining comparisons between motion estimate produced by the machine learning network with ground truth motion estimates.

A morphological processing unit 414 processes ultrasound datasets, which may comprise the source image dataset 402, consolidated data frames 406, or multi-scale datasets 408, 410. The morphological processing unit 414 estimates the properties of morphology of one or more data frames in the input dataset and differentiates properties of morphology likely to originate from a medical instrument relative to surrounding biological tissue, such as by differentiating one or more of: (a) intensity distribution, (b) directionality, (c) feature size, (d) connectedness, (e) edges, (f) blobs, (g) ridges, (h) corners, (i) combinations thereof, or other image features that would be understood to one practiced in the art. The morphological processing unit 416 may be analytical in nature or may be a machine learning network that is trained to estimate properties of morphology. The morphological processing unit 416 may be analytical in nature or may be a machine learning network that is trained to estimate properties of morphology. The trained machine learning model may be comprised of one or more generative adversarial networks (GANs), or any other machine learning model capable of generating 2D or 3D properties of morphology. The machine learning model architecture may comprise a U-Net, spatially aware autoencoders, context aware autoencoders, long-term short memory (LTSM), and/or recurrent neural networks (RNNs), as well as any other model architecture element advantageous for the purpose of generating estimates of properties of morphology from input images. In an exemplary embodiment, the machine learning model is trained through a generative adversarial network (GAN) architecture that generates estimates of properties of morphology from one or more input ultrasound images by iteratively refining comparisons between an estimate of properties of morphology produced by the machine learning network with ground truth estimates of properties of morphology.

A medical instrument likelihood estimation unit 416 receives the output of the motion processing unit 412 and the morphological processing unit 414 and determines the likelihood that pixels in the input datasets contain a medical instrument based on a weighted combination of the quantified motion and properties of morphology. In embodiments, the medical instrument likelihood estimation unit 416 preserves a memory of results from prior data frames to determine a likelihood the medical instrument exists within pixels of the current data frame. In an embodiment, the medical instrument likelihood estimation unit 416 comprises a state estimation technique, such as a Kalman filter or other approaches that would be understood to one practiced in the art, to predict the likelihood one or more pixels in the data frame contains the medical instrument based on a combination of a current state and one or more previous states. The medical instrument likelihood estimation unit 416 may be analytical in nature or may comprise a machine learning network that receives as input one or more of: (a) the output of the morphological processing unit 414, (b) the motion processing unit 412, (c) the sequence of ultrasound images, and (d) combinations thereof. The morphological processing unit 414 may be analytical in nature or may be a machine learning network that is trained to estimate medical instrument likelihood estimates. The trained machine learning model may be comprised of one or more generative adversarial networks (GANs), or any other machine learning model capable of generating 2D or 3D medical instrument likelihood estimates. The machine learning model architecture may comprise a U-Net, spatially aware autoencoders, context aware autoencoders, long-term short memory (LTSM), and/or recurrent neural networks (RNNs), as well as any other model architecture element advantageous for the purpose of generating medical instrument likelihood estimates from input images. In an exemplary embodiment, the machine learning model is trained through a generative adversarial network (GAN) architecture that generates medical instrument likelihood estimates from one or more inputs by iteratively refining comparisons between medical instrument likelihood estimates produced by the machine learning network with ground truth medical instrument likelihood estimates.

A medical instrument enhancement unit 418 receives the output of the medical instrument likelihood estimation unit 416 and alters the image to increase visibility of the medical instrument within the image during display. In preferred embodiments, altering the image comprises one or more of: (a) adjusting the intensity, (b) the hue, (c) the saturation, (d) the luminance, or (e) combinations thereof, of one or more pixels in the image containing the medical instrument relative to pixels containing surrounding biological tissue. In embodiments, the alteration comprises replacing the region containing the pixels likely to contain the medical instrument with a graphical representation of the medical instrument. The enhanced image produced by the medical instrument enhancement unit 418 is transferred to a display unit 420 to convey procedure guidance to an operator.

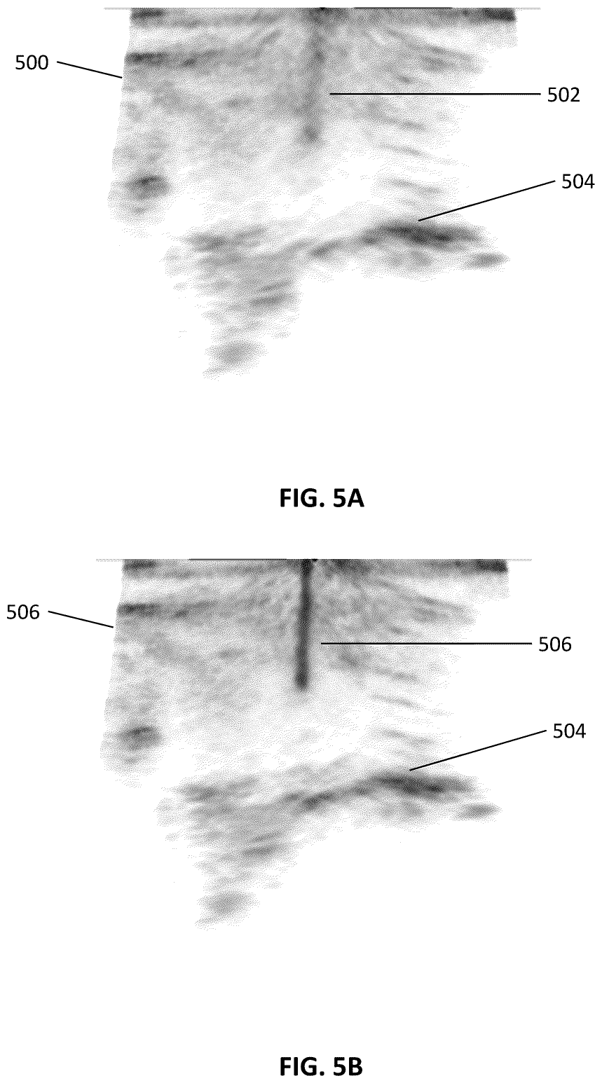

FIG. 5A depicts an exemplary non-enhanced ultrasound image 500 produced from an ultrasound system operating according to the embodiments and containing a dual-array ultrasound probe with a central gap for mid-line placement of a medical instrument. A medical instrument inserted through the central gap produces a region of pixels corresponding to the medical instrument 502 that is partially distinguished against surrounding biological tissue, which contains areas of similar or greater intensity 504 corresponding to bright connective tissue, tissue interfaces, or bony surfaces. FIG. 5B depicts an exemplary enhanced ultrasound image 506 produced from an ultrasound system operating according to the current embodiments and containing a dual-array ultrasound probe with a central gap for mid-line placement of a medical instrument. A medical instrument inserted through the central gap produces a region of pixels corresponding to the medical instrument 506 that has been detected and enhanced according to the methodology described herein and is well-distinguished against surrounding biological tissue.

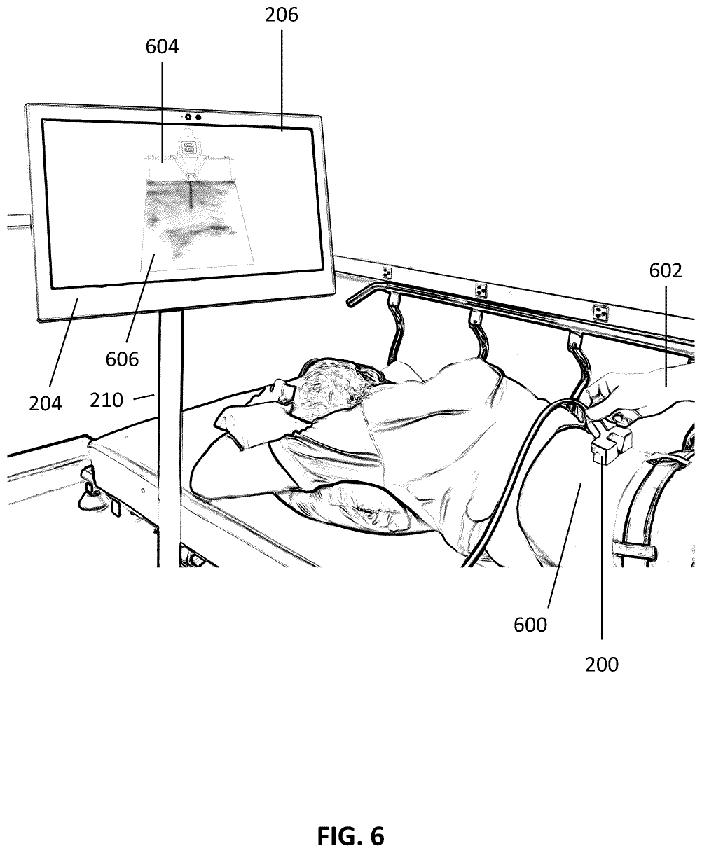

In one embodiment, an ultrasound system comprising a dual-array ultrasound probe 200, a computing unit 204 containing a processing unit, and a display unit 206 is depicted in FIG. 6. An operator 602 positions the probe against the patient anatomy 600 while the patient is in the prone position to execute a medical procedure involving a medical instrument to be inserted in the patient anatomy 600. The display unit 206 contains a composite image comprised of a graphical representation of the ultrasound probe 604 and an ultrasound image 606 for the purpose of guiding the medical procedure. The graphical representation of the ultrasound probe 604 and the ultrasound image 606 are altered to reflect the position of the probe relative to the operator so as to provide an intuitive representation of the medical instrument orientation during the procedure. In embodiments, the orientation of the composite image may be determined by user input, such as through a selection on a graphical user interface, by a button press on the ultrasound system or ultrasound probe, or through voice-activated commands. In embodiments, the orientation of the composite image may be determined by sensors embedded in the ultrasound probe housing and/or through optical sensors on the ultrasound system.

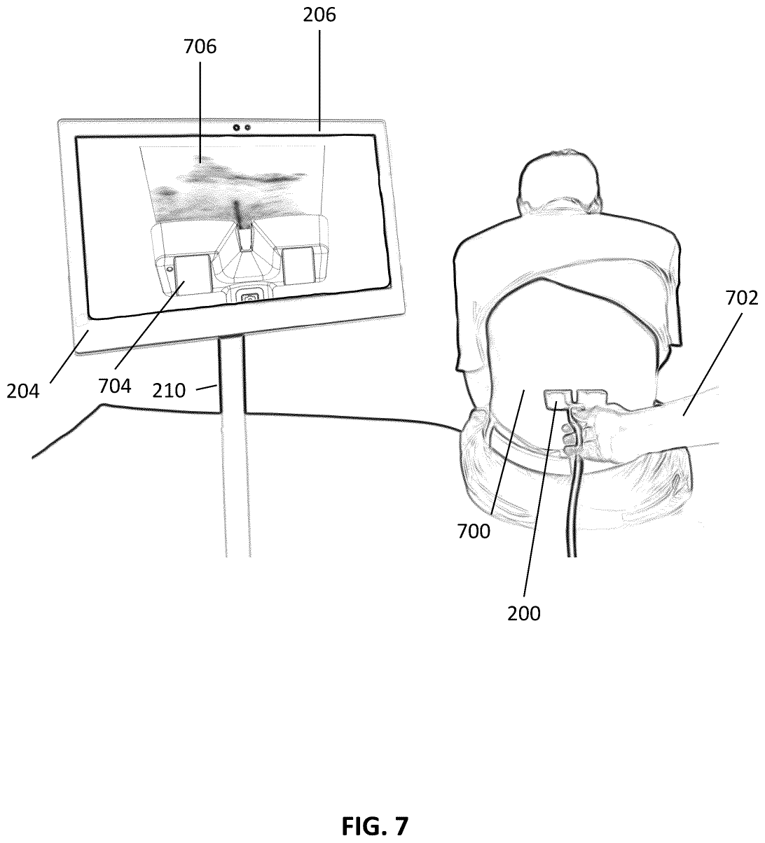

In one embodiment, an ultrasound system comprising a dual-array ultrasound probe 200, a computing unit 204 containing a processing unit, and a display unit 206 is depicted in FIG. 7. An operator 702 positions the probe against the patient anatomy 700 while the patient is in the seated position to execute a medical procedure involving a medical instrument to be inserted in the patient anatomy 700. The display unit 206 contains a composite image comprised of a graphical representation of the ultrasound probe 704 and an ultrasound image 706 for the purpose of guiding the medical procedure. The graphical representation of the ultrasound probe 704 and the ultrasound image 706 are altered to reflect the position of the probe relative to the operator so as to provide an intuitive representation of the medical instrument orientation during the procedure. In embodiments, the orientation of the composite image may be determined by user input, such as through a selection on a graphical user interface, by a button press on the ultrasound system or ultrasound probe, or through voice-activated commands. In embodiments, the orientation of the composite image may be determined by sensors embedded in the ultrasound probe housing and/or through optical sensors on the ultrasound system.

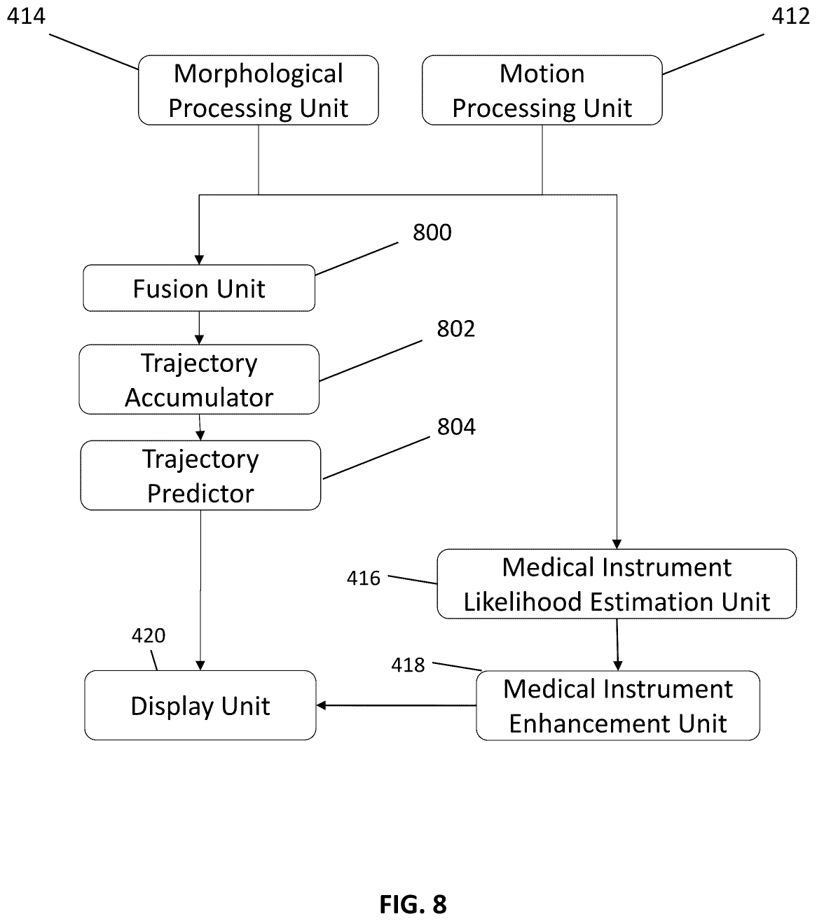

FIG. 8 depicts a block diagram describing the morphological processing unit 414, motion processing unit 412, medical instrument likelihood estimation unit 416, medical instrument enhancement unit 418, and display unit 420 of FIG. 4, and further comprises a fusion unit 800, trajectory accumulator 802, and trajectory predictor 804. In embodiments, the morphological and motion-derived measurements are combined to produce an improved measurement of the needle trajectory. The fusion unit 800 combines the quantified motion and properties of morphology from the motion processing unit 412 and the morphological processing unit 414, respectively, to generate a mathematical representation of the medical instrument's possible positions within an ultrasound data frame. The trajectory accumulator 802 processes the possible positions of the medical instrument to determine the most likely trajectory of the medical instrument relative to the ultrasound probe interface, or relative to an anatomical point of interest within the patient anatomy relevant to the interventional procedure, or combinations thereof.

In an embodiment, an integration of the observed motion may be computed for a set of linear paths which represent candidate needle trajectories. These linear paths may be constrained using the a priori knowledge ascribed by use of a medical instrument guide. Subsequently, the analyzed motion can be compared to a set of criteria to discriminate the presence of a medical instrument from other sources. For example, the criteria may include the average integrated motion, which can be used to distinguish the presence of global motion. If the criteria are met, this process yields a motion-estimated medical instrument trajectory. Alternatively, the needle trajectory may be estimated by a machine learning network that may include, by way of example, inputs of estimated motion and a priori knowledge ascribed by use of the medical instrument guide.

In embodiments, an estimate of the needle trajectory may be produced via quantifying properties of morphology of the medical instrument. In embodiments, a priori knowledge of the medical instrument provides a mathematical model of the expected appearance, such as a line segment with a given width for a medical needle. In embodiments, the trajectory accumulator 802 processes the quantified properties of morphology relative to a model of expected properties of morphology and calculates the most likely trajectory of the medical instrument.

A trajectory predictor unit 804 processes one or more calculated trajectories from the trajectory accumulator unit 802 to estimate a future location of the medical instrument relative to the ultrasound probe interface, or relative to an anatomical point of interest within the patient anatomy relevant to the interventional procedure, or combinations thereof. In embodiments the trajectory predictor unit 804 comprises a Kalman filter. The medical instrument trajectory is modelled as a linear time-variant function, e.g. the state-transition model, in which the trajectory is assumed to be a function of the current data frame's position in a sequence of data frames. A series of successive medical instrument trajectory measurements serve as the input to the trajectory predictor unit 804. In embodiments, one or more machine learning networks are used to estimate a future location of the medical instrument relative to the ultrasound probe interface, or relative to an anatomical point of interest within the patient anatomy relevant to the interventional procedure, or combinations thereof.

The display unit 420 combines one or more of the outputs of: (a) the medical instrument enhancement unit 418, (b) the trajectory accumulator 802, and (c) the trajectory predictor unit 804 to alter the output image for the purpose of procedure guidance. In embodiments, the calculated trajectory is compared with an estimated depth to an anatomical target and/or an ideal trajectory to an anatomical target to provide a display indication of medical instrument insertion progress. In embodiments, the calculated trajectory and/or predicted future trajectory are compared to points of interest relative to the ultrasound probe interface, or relative to an anatomical point of interest within the patient anatomy relevant to the interventional procedure, or combinations thereof, to provide a display indication of medical insertion progress.

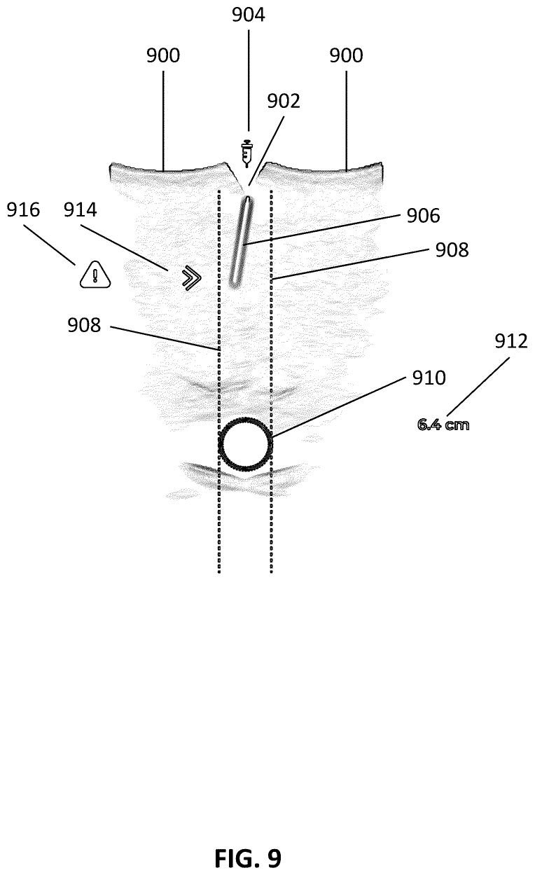

FIG. 9 depicts an exemplary ultrasound image with display indicators to convey progress of the medical instrument procedure on the basis of the invention described herein. A central medical instrument notch 902 can be visualized relative to the interface of two ultrasound arrays 900. A graphical representation of a medical instrument 904 is displayed to indicate the entry point of the medical instrument. A graphical representation of the medical instrument 906 replaces the pixels likely to contain the medical instrument on the basis of quantification of motion and properties of morphology relative to surrounding biological tissue. Indications of trajectory bounds 908 indicate a central acceptance region for an ideal medical instrument trajectory. A target indicator 910 indicates the location of the anatomical feature targeted by the procedure. A depth indicator represents the depth or distance from the probe interface to the target indicator, or may represent the depth or distance from the medical instrument tip to the target indicator. One or more trajectory bounds warning indicators 914, 916 indicate to the user that the medical instrument trajectory and/or a predicted future medical instrument location exceed the trajectory bounds indicators 908. In embodiments, the trajectory bounds warning indicators 914, 916 and trajectory bounds indicators 908 are altered depending on a degree of error between an ideal trajectory and the calculated trajectory and/or predicted future location of the medical instrument. For example, the trajectory bounds indicators 908 may change color when the medical instrument trajectory deviates from an ideal trajectory. Other variations from the examples described above will be readily apparent to those skilled in the art.

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

As used herein, the term “medical instrument” refers to a needle, catheter, or similar rigid and elongated tool intended to inject therapeutics or aspirate biological tissue.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. An ultrasound imaging and medical instrument guidance system comprising:
an ultrasound probe configured to transmit and receive acoustic signals from two or more ultrasonic arrays for generating a sequence of ultrasound images, wherein the two or more ultrasonic arrays are separated by a physical gap of at least 1 mm, said gap positioned to allow for in-plane insertion of the medical instrument relative to an ultrasound imaging plane, and wherein the gap is dimensioned to accommodate the insertion of the medical instrument into a patient's anatomy;
a display unit configured to produce a real-time or substantially real-time ultrasound image to provide visual feedback to an operator; and
a processor, and a storage having encoded thereon executable instructions that, when executed by the processor, cause the processor to carry out:
perform image and signal processing to reconstruct a sequence of ultrasound images from each of the two or more ultrasonic arrays;
perform image and signal processing to quantify one or more of the following from the sequence of ultrasound images: (a) a relative motion of the medical instrument compared to surrounding biological tissue, (b) properties of morphology of the medical instrument relative to surrounding biological tissue, (c) a combination of relative motion and properties of morphology of the medical instrument relative to surrounding biological tissue;
from an output of the image and signal processing, determine a likelihood that one or more pixels in the sequence of ultrasound images corresponds to the medical instrument; and
alter one or more of the following for the pixels determined to likely correspond to the medical instrument: (a) an intensity relative to surrounding biological tissue, (b) a hue relative to surrounding biological tissue, (c) a saturation relative to surrounding biological tissue, or (d) a luminance relative to surrounding biological tissue.

2. The system of claim 1, wherein the processor is further operative to carry out: quantify a predicted trajectory of the medical instrument based one or more of: (a) a quantification of the relative motion of the medical instrument compared to surrounding biological tissue, (b) one or more properties of morphology of the medical instrument relative to surrounding biological tissue, or (c) a combination thereof.

3. The system of claim 2, wherein the processor is further operative to carry out: measure one or more of: (a) a distance, (b), an angle, or (c) an error between the predicted trajectory of the medical instrument and a designated anatomical region.

4. The system of claim 1, wherein the processor is further operative to carry out: inform the operator of one or more of the following: (a) a position of the medical instrument within the sequence of ultrasound images based on the pixel likelihood determination, (b) a calculated trajectory of the medical instrument based on the pixel likelihood determination, or (c) a comparison of a predicted trajectory of the medical instrument to a planned trajectory of the medical instrument.

5. The system of claim 4, wherein informing the operator includes conveying to the operator whether the medical instrument's trajectory is colinear or misaligned with a designated anatomical location visualized within said sequence of ultrasound images.

6. The system of claim 5, wherein informing the operator includes providing a visual indication, and wherein an appearance of the visual indication is altered based on a calculation of misalignment of the medical instrument's trajectory with the designated anatomical location.

7. The system of claim 1, wherein said ultrasound probe comprises a dual-array geometry that enables a steep-angle medical instrument insertion through a center of a silhouette of the ultrasound probe.

8. The system of claim 7, further comprising an affixed apparatus to constrict movement of the medical instrument to a trajectory that spans between −30 and 30 degrees relative to a centerline of the ultrasound probe.

9. The system of claim 1, wherein the processor is further operative to carry out: operate one or more machine learning networks that are trained to quantify one or more of: (a) relative motion of the medical instrument compared to surrounding biological tissue, (b) properties of morphology of the medical instrument relative to surrounding biological tissue, or (c) combinations thereof.

10. The system of claim 1, wherein the processor is further operative to carry out: operate one or more machine learning networks that are trained to estimate a trajectory and/or a predicted future location of the medical instrument.

11. The system of claim 1, wherein the two or more ultrasound arrays are configured to emit high intensity ultrasound that generates sufficient acoustic radiation force to generate biological tissue motion.

12. The system of claim 1, wherein an external vibration source generates biological tissue motion.

13. The method of claim 1, wherein the ultrasound probe is configured to steer transmitted ultrasound energy from each of the two or more ultrasound arrays along a sequence of two or more unique angles to quantify a relative motion of the medical instrument, one or more morphological properties of the medical instrument, or combinations thereof.

14. A method for determining a likelihood that one or more pixels in a plurality of ultrasound images corresponds to a medical instrument, said method comprising the steps of:
acquiring the plurality of ultrasound images from an ultrasound probe configured to transmit and receive acoustic signals from two or more ultrasonic arrays that visualize an anatomical region where the medical instrument is to be inserted;
providing a processor and a storage having encoded thereon executable instructions that, when executed by the processor, cause the processor to carry out:
quantify one or more of: (a) motion of the medical instrument between two or more of the plurality of ultrasound images acquired by the two or more ultrasonic arrays, (b) one or more properties of morphology of the medical instrument in one or more of the plurality of ultrasound images acquired by the two or more ultrasonic arrays, or (c) combinations thereof; and
determine a likelihood that one or more pixels in one or more of the plurality of ultrasound images acquired by the two or more ultrasonic arrays represents or correspond to the medical instrument based on one or more of: (a) motion of the medical instrument relative to surrounding biological tissue, (b) one or more properties of morphology of the medical instrument relative to surrounding biological tissue, (c) spatial relationships to a geometry of the two or more ultrasonic arrays, or (d) combinations thereof.

15. The method of claim 14, wherein the two or more ultrasonic arrays are separated by a physical gap of at least 1 mm to provide for insertion of the medical instrument with an in-plane orientation relative to an ultrasound imaging plane.

16. The method of claim 15, further comprising an affixed apparatus to guide the medical instrument, wherein the affixed apparatus guides the medical instrument along an entry angle that spans between −30 and 30 degrees relative to a centerline of the plurality of ultrasound images.

17. The method of claim 14, wherein the quantified motion, or the one or more quantified properties of morphology, or a combination thereof, are used to quantify a predicted trajectory of the medical instrument within an imaging plane relative to the ultrasound probe.

18. The method of claim 17, wherein quantifying the predicted trajectory of the medical instrument comprises integrating a motion of the medical instrument measured along a series of paths that are a function of a geometry and/or electronic configuration of the ultrasound probe.

19. The method of claim 17, wherein one or more machine learning networks are trained to quantify the predicted trajectory of the medical instrument.

20. The method of claim 17, wherein a predictive engine estimates the predicted trajectory of the medical instrument based on one or more trajectories measured from one or more of the plurality of ultrasound images.

21. The method of claim 20, wherein one or more machine learning networks are trained to estimate the predicted trajectory of the medical instrument.

22. The method of claim 14, wherein one or more machine learning networks are trained to quantify from the plurality of ultrasound images one or more of: (a) the motion between two or more of the plurality of ultrasound images, (b) a relative motion of the medical instrument compared to surrounding biological tissue, (c) the one or more properties of morphology of the medical instrument in one or more of the plurality of ultrasound images, (d) properties of morphology of the medical instrument relative to surrounding biological tissue, (e) the likelihood that one or more pixels in the plurality of ultrasound images represents or corresponds to the medical instrument, or (f) combinations thereof.

23. The method of claim 14, wherein the motion of the medical instrument is quantified using a motion estimation based on spatiotemporally related pixels that are assumed to exhibit an apparent movement between at least two ultrasound images of the plurality of ultrasound images.

24. The method of claim 14, wherein the motion of the medical instrument is quantified by comparing a location of the medical instrument between two or more ultrasound images of the plurality of ultrasound images, wherein a difference in a time of acquisition of the two or more ultrasound images is at least twice of a period between sequential images of the plurality of ultrasound images, or wherein the motion of the medical instrument is quantified by comparing a location of the medical instrument between two or more ultrasound images of the plurality of ultrasound images, wherein the two or more ultrasound images are not sequential in the plurality of ultrasound images.

25. The method of claim 14, wherein the motion of the medical instrument is quantified between two or more ultrasound images of the plurality of ultrasound images using a multiscale image fusion process.

26. The method of claim 14, wherein one or more of the following is quantified from each of the two or more ultrasonic arrays independently: (a) the motion of the medical instrument, (b) the one or more properties of morphology of the medical instrument, or (c) combinations thereof.

27. The method of claim 14, wherein the ultrasound probe is configured to electronically steer ultrasound energy along a sequence of two or more angles to quantify the motion of the medical instrument, the one or more morphological properties of the medical instrument, or combinations thereof, as a function of steering angle.

28. An ultrasound imaging system for guiding insertion of a medical instrument into a patient anatomy, the system comprising:
an ultrasound probe configured to transmit and receive acoustic signals from two or more ultrasonic arrays to generate a sequence of ultrasound images;
a processor, and a storage having encoded thereon executable instructions that, when executed by the processor, cause the processor to carry out:
receive and process the sequence of ultrasound images from each of the two or more ultrasonic arrays;
perform image and signal processing to determine a likelihood that one or more pixels in the sequence of ultrasound images corresponds to the medical instrument;
alter one or more of the following for the pixels determined to likely correspond to the medical instrument: (a) an intensity of the pixels relative to surrounding biological tissue, (b) a hue of the pixels relative to surrounding biological tissue, (c) a saturation of the pixels relative to surrounding biological tissue, or (d) a luminance of the pixels relative to surrounding biological tissue; and
receive information indicating a position of the ultrasound probe relative to an operator, the patient anatomy, or both;
a computerized display configured to receive and display one or more of: the sequence of ultrasound images, an indication of a location of the medical instrument, or a combination thereof, and wherein said computerized display is further configured to display:
a graphical representation of the ultrasound probe, said representation being rotatable on the computerized display to reflect a position of the ultrasound probe relative to the operator, the patient anatomy, or both; and
one or more of the sequence of ultrasound images, said images being rotatable on the computerized display to reflect an orientation of the anatomy represented by the one or more sequence of ultrasound images relative to the operator, the patient anatomy, or both.

29. The system of claim 28, wherein the system further displays on the computerized display a medical instrument indication, said indication including at least one of (a) a position of the medical instrument within the sequence of ultrasound images, (b) a predicted trajectory of the medical instrument within the sequence of ultrasound images, (c) a comparison of the predicted trajectory to a planned trajectory of the medical instrument, or (d) a combination thereof.

30. The system of claim 28, wherein the processor, and a storage having encoded thereon executable instructions that, when executed by the processor, cause the processor to further carry out: provide real-time or substantially real-time feedback to the operator by adjusting a graphical representation of the medical instrument indication based on detected deviations from a planned trajectory.

31. The system of claim 28, wherein the processor, and a storage having encoded thereon executable instructions that, when executed by the processor, cause the processor to further carry out: provide haptic feedback to the operator through the ultrasound probe when deviations from a planned trajectory are detected.

32. The system of claim 29, wherein the computerized display is a touchscreen, allowing the operator to manually adjust a rotation or a zoom of one or more of: (a) one or more ultrasound images of the sequence of ultrasound images, (b) a representation of the ultrasound probe, (c) a medical instrument indication, or (d) combinations thereof.

33. The system of claim 28, wherein the processor, and a storage having encoded thereon executable instructions that, when executed by the processor, cause the processor to further carry out: receive input from one or more sensors tracking physical movement of the ultrasound probe and adjust the computerized display in real-time or substantially real-time based on changes in a position and/or orientation of the ultrasound probe.
